(19) United States
(12) Patent Application Publication
(10) Pub. No.: US 2015/0088197 A1
(43) Pub. Date: Mar. 26, 2015

(54) Bone Anchor Inserter

(71) Applicant: DePuy Mitek, LLC, Raynham, MA (US)

(72) Inventors: David B. Spenciner, North Attleboro, MA (US); Mark W. Wolfson, Wellesley, MA (US)

(21) Appl. No.: 14/036,469

(22) Filed: Sep. 25, 2013

(51) Int. Cl. A61B 17/04

(52) U.S. Cl. CPC A61B 17/0401; A61B 2017/0409
USPC 606/232

(57) ABSTRACT

Methods and devices are provided for deploying a bone anchor into bone. An inserter for deploying a bone anchor into bone can have an elongate shape with a proximal portion configured to be grasped by user and a distal tip configured to releasably mate with a bone anchor. In certain aspects, a distal portion of the inserter can be more flexible than remaining portions of the inserter and this can facilitate deploying the anchor into a bone tunnel. For example, as the bone anchor is being inserted into the bone tunnel, the distal portion of the inserter can be configured to bend to align the anchor with the tunnel. In one embodiment, the distal portion of the inserter has a plurality of grooves spaced along the inserter that define a series of rings such that when the inserter bends, the adjacent rings contact one another to limit the applied strain.

### FIELD

The present disclosure relates to devices and methods for deploying a bone anchor into bone.

### BACKGROUND

The complete or partial detachment of ligaments, tendons and/or other soft tissues from their associated bones within the body are relatively commonplace injuries, particularly among athletes. Such injuries are generally the result of excessive stresses being placed on these tissues. By way of example, tissue detachment may occur as the result of an accident such as a fall, over-exertion during a work-related activity, during the course of an athletic event, or in any one of many other situations and/or activities. In the case of a partial detachment, the injury will frequently heal itself, if given sufficient time and if care is taken not to expose the injury to further undue stress. In the case of complete detachment, however, surgery may be needed to re-attach the soft tissue to its associated bone or bones.

Numerous devices are currently available to re-attach soft tissue to bone. Examples of such currently-available devices include screws, staples, suture anchors, and tacks. In soft tissue re-attachment procedures utilizing screws, the detached soft tissue is typically moved back into its original position over the bone. Then the screw is screwed through the soft tissue and into the bone, with the shank and head of the screw holding the soft tissue to the bone. Similarly, in soft tissue re-attachment procedures utilizing staples, the detached soft tissue is typically moved back into its original position over the bone. Then the staple is driven through the soft tissue and into the bone, with the legs and bridge of the staple holding the soft tissue to the bone.

In soft tissue re-attachment procedures utilizing suture anchors, an anchor-receiving hole is generally first drilled in the bone at the desired point of tissue re-attachment. A suture anchor is then deployed in the hole using an appropriate installation tool, commonly referred to as an inserter. When a knotless suture anchor is used, deploying the suture anchor into the hole effectively locks the suture, with the free end(s) of the suture extending out of the bone. The free ends of the suture are passed through or around soft tissue and are used to attach the soft tissue securely to the bone.

While there are various methods for attaching tissue to bone using a suture anchor, it can be challenging to deploy the suture anchor into a tunnel formed in the bone, especially when a knotless suture anchor is used. For example, after the tunnel is formed in the bone and the drill is removed from the surgical site, it can be difficult for a surgeon to locate the tunnel and navigate surrounding tissue and/or bone to gain access to the bone tunnel. Even after the location of the tunnel is identified, it can be difficult to determine the correct angle of the bone tunnel and to guide the suture anchor into the hole using an inserter. Often, forcing a suture anchor into the hole at an improper angle will generate high stresses on the anchor, causing it to fail.

Accordingly, there remains a need for improved methods and devices for deploying a bone anchor into bone and attaching soft tissue to bone.

### SUMMARY OF THE INVENTION

Various bone anchors, inserters, and methods for deploying a bone anchor into bone are provided herein. In one embodiment, an inserter for driving a suture anchor into bone has an elongate shaft with proximal and distal ends, and a flexible portion extending along a length thereof adjacent to the distal end. The flexible portion can include a plurality of grooves extending at least partially circumferentially around the shaft and spaced at a distance apart from one another along a longitudinal axis of the shaft. The inserter can also include a distal tip extending distally from the distal end of the elongate shaft. The distal tip of the inserter can be configured to mate with a suture anchor.

The inserter can vary in a number of ways. For example, the flexible portion of the inserter can include a plurality of rings extending at least partially circumferentially around the elongate shaft and defining the plurality of grooves therebetween. In certain aspects, the plurality of rings can have equal sized diameters. In other aspects, the plurality of rings can have diameters that vary in a proximal-to-distal direction. The spacing of the plurality of rings along the longitudinal axis of the shaft can also vary. In certain aspects, the plurality of rings can be spaced at equal distances apart along the longitudinal axis of the elongate shaft.

In some embodiments, the flexible portion of the elongate shaft can have a flexibility that varies in a proximal-to-distal direction. In certain aspects, a central longitudinal axis of the elongate shaft can be substantially aligned with a central longitudinal axis of the plurality of rings. In other aspects, the flexible portion of the shaft can have a diameter that varies from a proximal-to-distal direction. In certain aspects, an outer diameter of the distal tip can be less than an outer diameter of the elongate shaft such that the distal tip is configured to extend into an inner lumen in a suture anchor.

A suture anchor system is also provided herein that includes a suture anchor and an inserter. In certain aspects, the suture anchor has an elongate body with proximal and distal ends and an inner lumen extending at least partially therethrough. The inserter has an elongate shaft with proximal end and distal ends. The distal end of the inserter includes a tip portion configured to couple to the suture anchor to mate the suture anchor to the elongate shaft, and a flexible portion positioned proximal to the tip portion and configured to allow the elongate shaft to bend along the flexible portion during insertion of the suture anchor into bone.

The inserter and the suture anchor can have a variety of sizes, shapes, and configurations. In certain aspects, the inserter can have an inner lumen extending between the proximal end and the tip portion for receiving a suture therethrough. The flexible portion of the inserter can also vary. In one embodiment, the flexible portion of the inserter includes a plurality of grooves spaced at a distance apart along the elongate shaft. In another embodiment, the flexible portion of the inserter includes a plurality of rings extending at least partially circumferentially around the elongate shaft and spaced apart along a longitudinal axis of the elongate shaft. In certain aspects, when the inserter is mated to the suture anchor, the flexible portion of the elongate shaft abuts a proximal end of the suture anchor. In some embodiments, the suture anchor can have a suture-engaging member adapted to receive a suture therearound.

A method for attaching tissue to bone is also provided herein and includes a method for deploying a bone anchor into bone. In one embodiment, a bone anchor is disposed around a distal tip of an inserter shaft and is inserted into a bone hole. The inserter can have a plurality of grooves that define a flexible region formed in a distal portion. The flexible region of the inserter is located proximal to the distal tip of the inserter and proximal to the suture anchor. When the suture anchor is inserted into the bone hole, the flexible region on the inserter can bend to help position the anchor in the bone hole. In an exemplary embodiment, the flexible region bends to allow a longitudinal axis of the bone anchor to align with a longitudinal axis of the bone hole.

The bone anchor can be deployed into the bone hole in various ways. For example, the bone anchor can be inserted into the bone hole without advancing the bone anchor over a guide wire. In other aspects, the bone anchor is inserted into the bone hole without inserting the bone anchor inside of a guide member. In one embodiment, the flexible region of the inserter shaft includes a plurality of rings having the grooves formed therebetween such that when the inserter bends along the flexible region, at least some of the plurality of rings contact one another to limit a strain applied to the inserter. In another embodiment, the inserter plastically deforms as the bone anchor is inserted into the bone hole. In yet another embodiment, the bone anchor is inserted into a glenoid rim.

### BRIEF DESCRIPTION OF DRAWINGS

This invention will be more fully understood from the following detailed description taken in conjunction with the accompanying drawings, in which:

FIG. 1A is a perspective view of an exemplary bone anchor system that includes a suture anchor and an inserter;

FIG. 1B is a cross-sectional, perspective view of the inserter of FIG. 1A taken along a longitudinal axis of the inserter;

FIG. 2 is a side view of the flexible portion of the inserter of FIG. 1A showing relevant dimensions that influence a bending angle between adjacent rings;

FIG. 3A is a side view of an exemplary inserter having a plurality of identical rings spaced along the longitudinal axis of the inserter;

FIG. 3B is a side view of yet another exemplary inserter that includes a plurality of rings having diameters that increase in the proximal-to-distal direction;

FIG. 3C is a side view of yet another exemplary inserter that includes a plurality of rings spaced at decreasing distances apart in the proximal-to-distal direction and having a shaft diameter that increases in the proximal-to-distal direction;

FIG. 3D is a side view of another exemplary inserter having a flexible portion with a shaft diameter that decreases in a proximal-to-distal direction;

FIG. 3E is a side view of another exemplary inserter having a plurality of rings, a longitudinal axis of one or more of the rings being offset from a longitudinal axis of the inserter;

FIG. 4 is a side view of another embodiment of an inserter having chamfers formed on the plurality of rings to facilitate insertion and removal of the inserter from a patient's body;

FIG. 5A is a side view of a distal tip of an inserter, according to one exemplary embodiment;

FIG. 5B is an end view of the distal tip of FIG. 5A;

FIG. 6A is a perspective view of one embodiment of the suture anchor;

FIG. 6B is a cross-sectional view of the suture anchor of FIG. 6A taken along a longitudinal axis of the anchor;

FIG. 7A is a perspective view of an exemplary inserter mated with a suture anchor;

FIG. 7B is a cross-sectional view of the inserter and suture anchor of FIG. 7A taken along a longitudinal axis of the inserter;

FIG. 8 is a perspective view of the inserter of FIG. 3A having the suture anchor of FIG. 6A coupled thereto, the inserter being in a straightened configuration;

FIG. 9 is a perspective view of the inserter and suture anchor of FIG. 8, the inserter being in a flexed or bent configuration guiding the suture anchor into bone; and

FIG. 10 is a perspective view of the inserter and suture anchor with the inserter being flexed in an opposite direction as in FIG. 9.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention. Further, in the present disclosure, like-numbered components of the embodiments generally have similar features, and thus within a particular embodiment each feature of each like-numbered component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

The figures provided herein are not necessarily to scale. Still further, to the extent arrows are used to describe a direction a component can be tensioned or pulled, these arrows are illustrative and in no way limit the direction the respective component can be tensioned or pulled. A person skilled in the art will recognize other ways and directions for creating the desired tension. Likewise, while in some embodiments movement of one component is described with respect to another, a person skilled in the art will recognize that other movements are possible. Additionally, a number of terms may be used throughout the disclosure interchangeably but will be understood by a person skilled in the art.

Methods and devices are provided for deploying a bone anchor into bone. In general, an inserter is provided having a generally elongate shape with a proximal portion configured to be grasped by user and a distal tip configured to releasably mate with a bone anchor. In certain aspects, a distal portion of the inserter can be more flexible than remaining portions of the inserter and this can facilitate deploying the anchor into a bone tunnel. For example, as the bone anchor is being inserted into the bone tunnel, the distal portion of the inserter can bend to align the anchor with the tunnel. The inserter can have a variety of sizes and shapes, and can be formed from various materials selected to achieve a desired flexibility. In one embodiment, the inserter can have a plurality of grooves spaced along the distal portion of the inserter so that as the inserter bends, adjacent projections contact one another to limit the applied strain and prevent the inserter from failing. In one embodiment, the inserter can include a series of rings spaced apart along the inserter and defining the grooves therebetween. The rings can be configured to contact one another during bending. In another embodiment, the flexibility of the distal portion of the inserter can be altered using various manufacturing processes and techniques, such as heat treatment and/or annealing. This flexible distal portion can allow the bone anchor to be deployed into bone that is not readily accessible through a straight, on-axis approach. In other words, a longitudinal axis of the bone anchor can align with a longitudinal axis of the bone hole while the inserter can extend off-axis. Such a configuration can be particularly advantageous where visibility is poor and the bone anchor is inserted blindly into the bone hole without the use of a guidewire or a guide device, or where insertion along a linear axis is otherwise difficult.

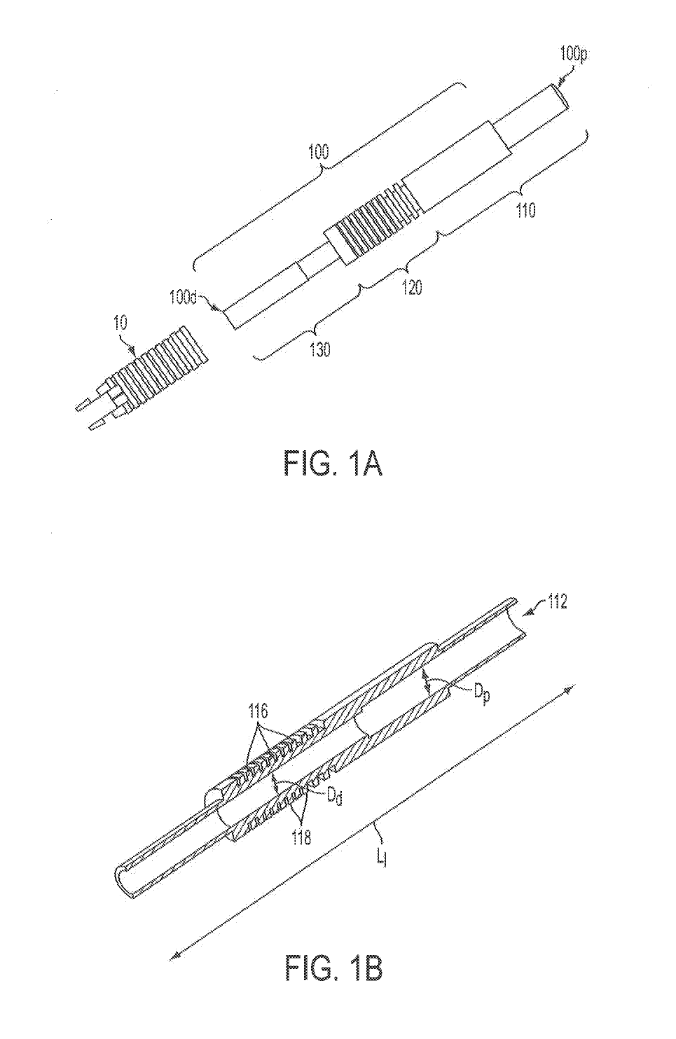

FIG. 1A is a perspective view of a suture anchor 10 and an inserter 100 configured to deploy the suture anchor 10 into bone, according to one exemplary embodiment. In general, the inserter 100 includes an elongate shaft having a proximal portion 110, a distal portion 120, and a distal tip 130 extending from the distal portion 120. As shown in FIG. 1B, the inserter 100 can have an inner lumen 112 formed therein that extends through the entire length thereof from a proximal end 100p to a distal end 100d of the inserter 100. The inner lumen 112 can be configured to receive legs of a suture (not shown) therethrough, as will be explained in greater detail below. The inserter 100 can alternatively have a solid structure along one or more portions of its length or the inserter 100 can be substantially solid along its entire length from the proximal end 100p to the distal end 100d. As will be appreciated by a person skilled in the art, the inner lumen 112 of a cannulated inserter can vary in any number of ways. For example, the inner lumen 112 can have substantially constant diameter along its length from the proximal end to the distal end, or the diameter of the inner lumen can increase or decrease from the proximal portion to the distal end. In the illustrated embodiment, a diameter D_{D} of the inner lumen 112 at the distal portion 120 is less than a diameter D_{P} of the lumen 112 at the proximal portion 110. In general, the inserter 100 can have a length L_{I} from the proximal end 100p to the distal end 100d sized such that the distal tip 130 and the distal portion 120 can be positioned inside a patient's body and at least a portion of the proximal portion 110 can extend outside a patient's body. The respective lengths of the various portions of the inserter 100 are not shown to scale in the figures.

The proximal portion 110 of the inserter 100 can have a variety of configurations. In one embodiment, the proximal portion 110 can include a handle (not shown) configured to be grasped and manipulated by a user. The handle can have a contoured shape that facilitates being manually grasped by a user. As will be appreciated by person skilled in the art, the handle can include one or more surface features that increase friction between a user's hand and the handle.

As previously mentioned, the inserter 100 can have a distal portion 120 that is more flexible than other portions of the inserter. The flexible distal portion 120 of the inserter can have a variety of sizes, shapes, and configurations, and can be positioned at various locations along the length of the inserter 100. In the illustrated embodiment, the flexible distal portion forms a region between the proximal shaft and the distal tip. In an exemplary embodiment, the flexible distal portion 120 is positioned immediately adjacent to and proximal of the distal tip 130. Such positioning can facilitate angulation of the tip 130 relative to the proximal portion 110 during use of the inserter, as discussed below. In another embodiment, the flexible distal portion need not be immediately adjacent to the distal tip and can instead be separated from the distal tip at any distance selected to achieve a desired bending location on the inserter. The length of the flexible distal portion 120 can also vary. For example, the flexible distal portion 120 can be about 0.01 to 10% of the total length L_{I} of the inserter 100, but is preferably 1% of the length L_{I}.

In certain aspects, the flexible portion 120 can include a plurality of grooves or cut-outs formed in the shaft that define adjacent projections that can interact with one another when the inserter bends, thus defining the distal flexible portion. The grooves can have a variety of shapes, including by way of non-limiting example, square, rectangular, and cylindrical. The grooves can be spaced apart at various discrete locations along a length and around a circumference of the elongate shaft such that the inserter has multiple favored bending axes. In the embodiment shown in FIGS. 1A and 1B, grooves 116 each have an annular shape such that they extend circumferentially around an outer surface of the shaft, forming a continuous shape. In general, three or more circumferential grooves 116 can be formed in and spaced along the longitudinal axis of the shaft and can define two or more cylindrical rings 118. The plurality of rings 118 can be integrally formed on the shaft using a variety of known techniques, as will be discussed in greater detail below. Alternatively, the plurality of rings 118 can be pre-formed and fixed along longitudinal axis of the shaft, but can be configured to rotate relative to the shaft. In embodiments where the inserter has an inner lumen formed therein, the plurality of grooves 116 can extend into an outer surface of the elongate shaft at a depth that avoids penetration of the inner lumen 112 of the shaft. In other words, the grooves 116 can have a depth that is less than a radius of the shaft, and more preferably that is less than the difference between the radius of the shaft and the radius of the inner lumen 112. The grooves can vary in other ways. For example, the grooves can extend partially around the circumference of the outer surface of the shaft, forming a non-continuous shape.

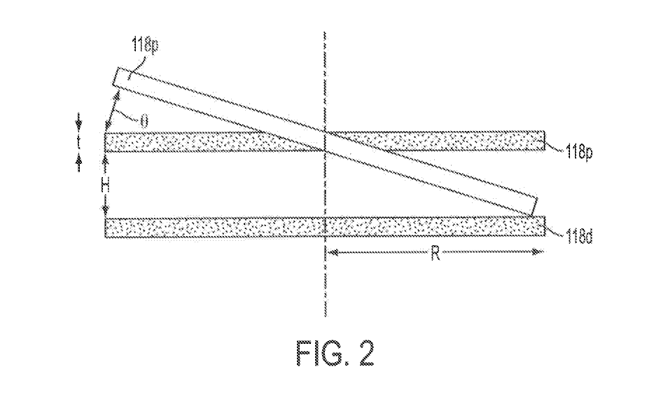

A bending angle between two adjacent rings can depend on dimensions of the rings and spacing between the adjacent rings. As shown in FIG. 2, two adjacent rings 118p, 118d will contact each other at a bending angle that depends on a distance separating the rings 118p, 118d, a thickness of the rings 118p, 188d, and a maximum radius R of distal ring 118d. More specifically, a bending angle θ can be calculated by the equation,

$$θ = a tan ( \frac{H}{R} ) ,$$

where H is a longitudinal distance separating the rings 118p, 118d and R is a maximum radius of the distal ring 118d Although the thickness T of the rings 118p, 118d is not provided in this equation, it can also affect the bending angle θ, as the rings 118p, 118d may flex under an applied load. Such flexing can be greater as the thickness T decreases and/or as the maximum radius R increases. As will be appreciated by a person skilled in the art, the distance H, the maximum radius R, and the thickness T can vary in any number of ways. In one embodiment, the distance H can be in the range of about 0.1 mm to 2 mm, but is preferably about 0.5 mm. The thickness T can be in the range of about 0.1 mm to 2 mm, but is preferably about 0.5 mm. The maximum radius R of the ring can be in the range of about 1 mm to 5 mm, but is preferably about 3 mm.

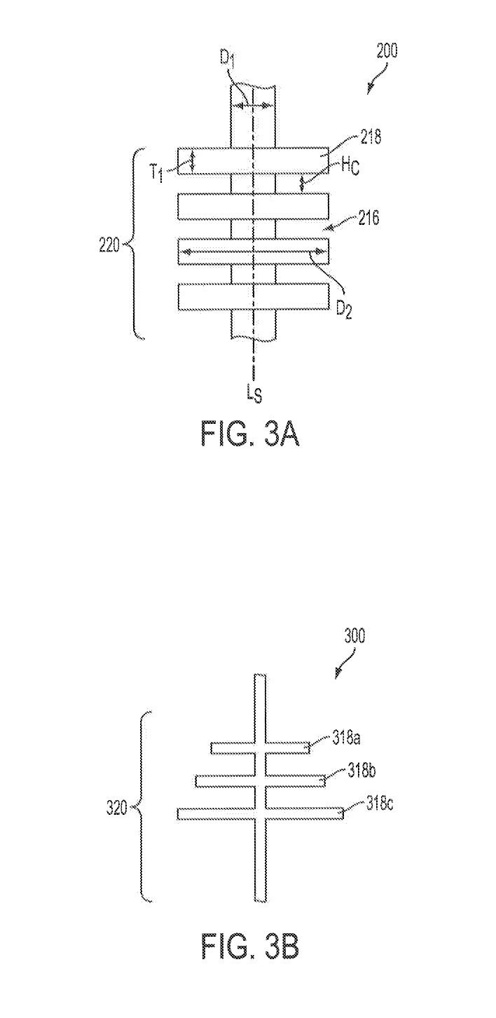

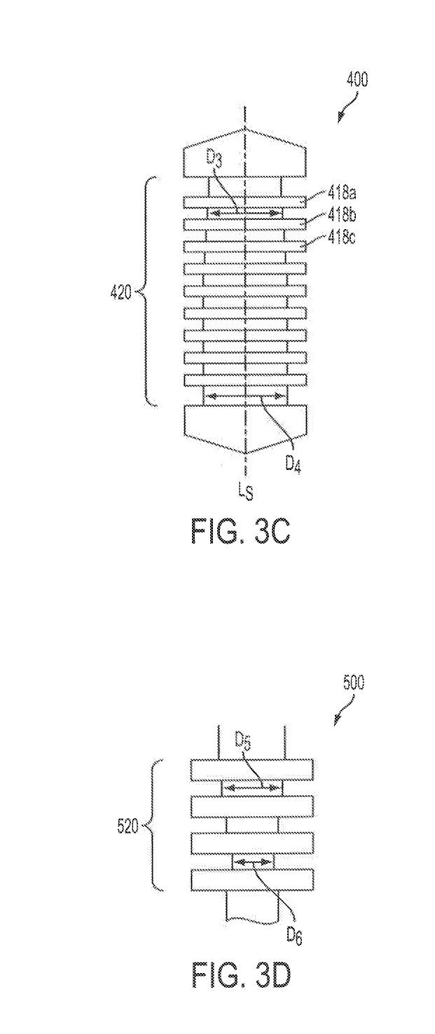

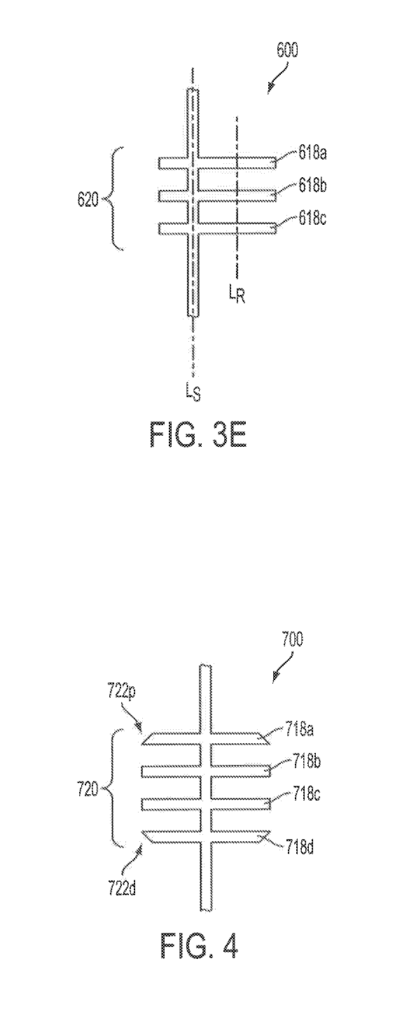

The flexible distal portion 120 of the inserter 100 can have various combinations of grooves, projections, and shaft diameters. A person skilled in the art will appreciate that the particular combination of rings and shaft diameters can be selected to achieve a desired bending angle θ, and that the combination of features are not limited to those shown in the figures. In one embodiment shown in FIG. 3A, an inserter 200 can have identical sized and shaped rings 218, and a constant shaft diameter D₁ along a distal portion 220 of the inserter 200. More specifically, the rings 218 can be symmetrical about a longitudinal axis L_{S} of the inserter 200 and a thickness T₁ of each of the plurality of rings 218 can be the same. Additionally, a diameter D₂ of each of the plurality of rings 218 can be the same, where the diameter D₂ is measured along an axis that is perpendicular to the longitudinal axis L_{S} of the inserter 200. The rings 218 can be spaced apart at a distance H_{C} defined by a height of each groove 216, and the spacing between the rings 218 and the height H_{C} of the grooves 216 can be the same along the proximal-to-distal length of the distal portion 220. The height H_{C} of the grooves 216 can be measured along the longitudinal axis of shaft L_{S}.

As shown in FIG. 3B, in another embodiment, an inserter 300 can have a flexible distal portion 320 with a plurality of rings 316 having sizes that are selected to influence bending of the flexible distal portion 320. More specifically, the flexible distal portion 320 can include rings 318a, 318b, 318c having diameters that increase in the proximal-to-distal direction so that when a bending force is applied to the inserter 300, bending of the inserter 300 progresses along the flexible distal portion 320 in a proximal-to-distal direction. That is, the flexible distal portion 320 bends first at a proximal end, such as via proximal ring 318a, and then continues to bend moving distally therealong to rings 318b and 318c. Such progressive bending will be discussed in greater detail with respect to the methods of deploying the suture anchor 10 into bone.

In another embodiment, a flexible distal portion of the inserter can have a shaft with a diameter that varies along the longitudinal axis of the inserter. For example, as shown in FIG. 3C, a flexible distal portion 420 can have a shaft with a diameter that increases in the proximal-to-distal direction along the longitudinal axis L_{S} of the inserter 400. As shown, a diameter D₃ at a proximal region of the flexible portion 420 can be less than a diameter D₄ at a distal region of the flexible portion 420 of the inserter. As shown, a height of each groove can vary along the longitudinal axis of the inserter. More specifically, a height of each of the grooves can decrease from the proximal-to-distal direction along the longitudinal axis of the inserter. This configuration can affect bending of the flexible distal portion 420. More specifically, a proximal-most ring 418a of the flexible distal portion will bend first and then distal rings 418b, 418c, etc. will bend, with bending progressing in the proximal-to-distal direction. Alternatively, as illustrated in FIG. 3D, an inserter 500 can have a shaft with a diameter that decreases in the proximal-to-distal direction along the longitudinal axis L_{S} of the inserter 500, where a diameter D₅ of a proximal portion of the shaft is greater than a diameter D₆ of a distal portion 520 of the shaft. A height of each groove can be substantially equal along the longitudinal axis of the inserter, as shown, or a height of each groove can increase or decrease from the proximal-to-distal direction.

In other embodiments, the inserter can have a flexible distal portion with various features that allow the inserter to have one or more favored bending directions. That is, the flexible distal portion of the inserter can be configured to achieve a higher angle of bending in one or more of the favored bending directions than if the inserter is bent in a non-favored direction. As in the embodiment shown in FIG. 3E, an inserter 600 can have a flexible distal portion 620 with a plurality of rings 618a, 618b, 618c having identical diameters and identical thicknesses and spaced at identical distances apart, but a central longitudinal axis L_{R} of one or more of the plurality of rings 618a, 618b, 618c can be offset from the central longitudinal axis L_{S} of the inserter 600. In these embodiments, the proximal portion (not shown) of the inserter can have a marking, surface feature, etc. on the handle of the inserter so that user can identify which direction(s) is the one or more favored bending directions. In the illustrated embodiment, the favored bending direction is on the left hand side of the inserter 600 because the plurality of rings 618a, 618b, 618c extend from the shaft at a distance that is smaller than at the right hand side of the device.

The flexible distal portion of the inserter can have a variety of other features. FIG. 4 illustrates an embodiment of an inserter having a plurality of rings 718a, 718b, 718c, 718d, where the rings 718a and 718b have chamfers that can facilitate insertion and removal of the inserter from a patient's body. In general, one or more rings positioned at a distal end of the flexible distal portion can have a distal chamfer in which a diameter of the ring gradually decreases in the proximal-to-distal direction to facilitate advancement of the inserter through the patient's body, toward the bone. As another example, or more rings positioned at a proximal end of the flexible distal portion can have a proximal chamfer in which a diameter of the ring gradually increases in the proximal-to-distal direction to facilitate removal of the inserter from the patient's body after the anchor has been deployed from the inserter. A person skilled in the art will appreciate that any number of rings or projections on the flexible distal portion can have a proximal chamfer and/or any number of rings or projections can have a distal chamfer. In the illustrated embodiment, proximal ring 718a has a proximal chamfer 722p formed on an outer-facing surface thereof and distal ring 718d has a distal chamfer 722d formed on another outer-facing surface thereof.

The flexible distal portion of the inserter can be formed from a variety of different materials. Depending in part on the type of material that forms the inserter, the inserter can undergo elastic, non-elastic, or plastic deformation. By way of non-limiting example, the inserter can be formed from a shape memory material such as Nitinol that bends back to the straightened configuration when a bending force is no longer applied. As another example, the inserter can be formed from a metal, such as stainless steel, that can bend back to an original straightened configuration or can undergo plastic deformation. A person skilled in the art will appreciate that the inserter can be formed from one or more materials that can be selected so that the inserter achieves elastic or plastic deformation. For example, as will be appreciated by a person skilled in the art, the plurality of rings can be formed from the same material as the shaft or from one or more different materials than the shaft material. As another example, the material can be altered during manufacturing to achieve a desired bending quality, such as via heat treatment and/or annealing. More specifically, the heat treatment and/or annealing can be applied at a higher level on the proximal portion of the flexible portion, and lesser amounts of heat treatment and/or annealing can be applied toward the distal portion. This can create a gradual increase in flexibility along a length of the distal portion or can create a stepped increase in flexibility depending on the amount of heat treatment and/or annealing and the locations applied to the shaft.

As will be appreciated by a person skilled in the art, the grooves can be formed along the flexible portion of the inserter using a variety of manufacturing techniques. In one embodiment, material can be removed from an outer surface of a shaft to define grooves in the inserter shaft. These grooves can define a plurality of rings, as previously discussed. In another embodiment, the plurality of rings can be attached to a shaft during manufacturing, such as via welding or other mating techniques known in the art.

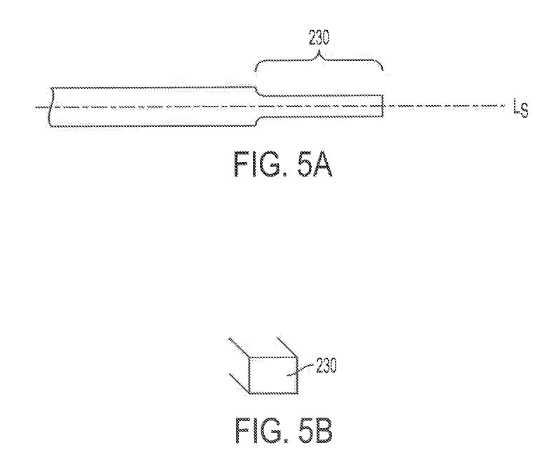

The distal tip of the inserter can have various sizes, shapes, and configurations, and can be configured to releasably mate with a suture anchor in various ways. In one embodiment the distal tip of the inserter can have a size and shape such that it is configured to mate with an outer surface of the suture anchor, such as by sliding partially over an outer surface of the proximal end of the suture anchor. In another embodiment, when the releasable mating occurs via an inner lumen of a suture anchor, a size and shape of the distal tip of the inserter can substantially match or correspond to a size and shape of the inner lumen of the suture anchor. By way of non-limiting example, a distal tip of the inserter can have a cross-sectional shape that can be circular, triangular, hexagonal, etc., and the inner lumen can have a cross-sectional shape that is identical to the cross-sectional shape of the distal tip of the inserter. The distal tip of the inserter can alternatively or additionally be tapered in the proximal-to-distal direction, and the degree of tapering of the inserter can substantially correspond to a tapering of the inner lumen of the suture anchor 10 in the proximal-to-distal direction. FIGS. 5A and 5B illustrate one embodiment of a distal tip 230 having a rectangular cross-sectional shape with a constant diameter and no tapering along the longitudinal axis L_{S} of the inserter. As will be appreciated by a person skilled in the art, the releasable mating can occur as an interference or press fit, as a rotational fit via matching threads on the inner lumen of a suture anchor and on the distal tip of the inserter, or via tensioning suture to retain the anchor on the distal tip of the inserter. As another example, an inner lumen of a suture anchor can include one or more surface features that can increase friction between the distal tip of the inserter and the inner lumen of the suture anchor to facilitate releasable mating of the inserter and the suture anchor.

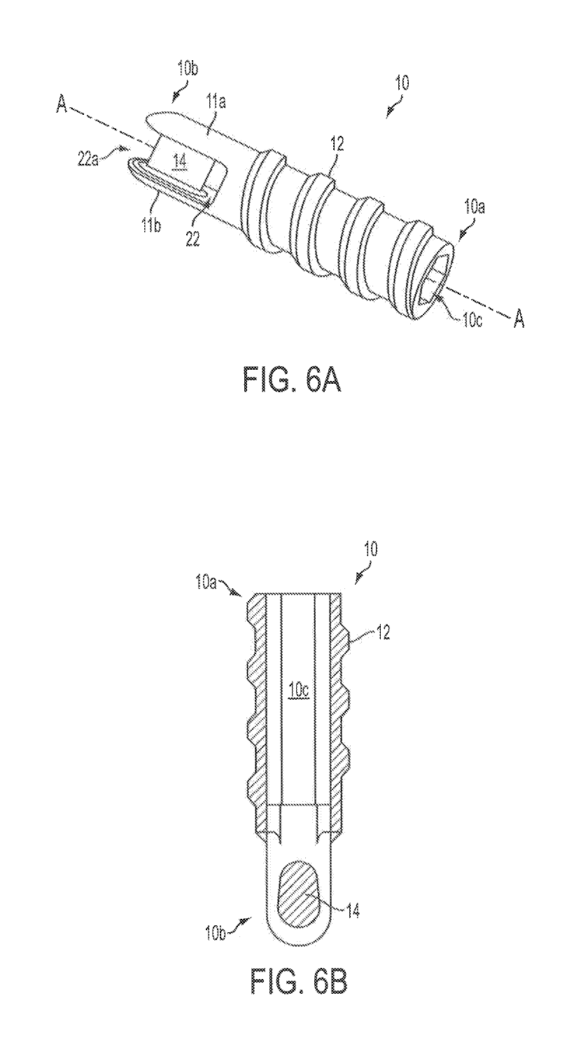

The inserters provided herein can be configured to mate with suture anchors having various features and configurations. FIGS. 6A-6B illustrate the exemplary suture anchor 10 of FIG. 1A in greater detail. As shown, the suture anchor 10 is in the form of a generally elongate body having proximal and distal ends 10a, 10b with an inner lumen 10c extending therethrough. At least one bone-engaging surface feature 12 can be formed on at least a portion of an external surface thereof for engaging bone. The suture anchor 10 also includes a suture-engaging member 14 disposed distal to the inner lumen 10c adjacent to the distal end 10b of the suture anchor 10. The suture-engaging member 14 is adapted to receive one or more sutures (two sutures 16, 18 are shown) therearound such that the suture(s) can extend around the suture-engaging member 14 and trailing ends of the suture(s) can extend through the inner lumen 10c and out of the proximal end 10a of the suture anchor 10. Exemplary suture anchors are shown, for example, in U.S. Pat. No. 8,114,128 filed on Nov. 1, 2006 and entitled “Cannulated Suture Anchor,” which is hereby incorporated by reference in its entirety.

The body of the suture anchor 10 can have a variety of configurations, shapes, and sizes. In an exemplary embodiment, the body is configured to be implanted within a bone tunnel formed in bone, and more preferably it has a size and shape that allows it to be fully engaged through the thickness of the cortical bone. In the illustrated embodiment the body has a generally elongate cylindrical shape with a blunt or rounded distal end 10b to facilitate introduction into a bone tunnel. The proximal end 10a of the body is head-free, as the cannulated configuration of the body allows an inserter to be inserted into the inner lumen 10c to drive the suture anchor 10 into bone. As indicated above, the suture anchor 10 can also include one or more bone-engaging surface features formed thereon and adapted to engage bone. While various surface features can be used, such as teeth, ridges, protrusions, etc., in an exemplary embodiment the body can include one or more threads extending therearound. In the illustrated embodiment a single thread extends around the body from the proximal end 10a and it terminates proximal to the distal end 10b. The particular location at which the thread terminates can vary depending on the particular configuration of the suture anchor 10. As will be discussed in more detail below, the illustrated suture anchor 10 can include opposed cut-outs formed in the distal end thereof and the thread can terminate just proximal to the proximal end of the cut-outs.

The suture anchor 10 can also be formed from a variety of materials. In an exemplary embodiment, the material has physical properties that are sufficient to allow an inserter to be inserted into the inner lumen 10c of the suture anchor 10 and to be used to drive the suture anchor 10 into bone without damaging the suture anchor 10. The properties of the material will of course depend on the particular configuration of the suture anchor 10. For example, the inner lumen 10c of the suture anchor 10 can have a length that maximizes the torque strength of the suture anchor 10 as well as the amount of surface contact between an inserter and the suture anchor 10, thus allowing weaker materials, such as bioabsorbable and/or osteoconductive materials to be used. A person skilled in the art will appreciate that a variety of other materials, including plastics and metals, can be used to form the suture anchor 10.

As previously indicated above, the suture anchor 10 can also include a suture-engaging member 14 formed therein. The suture-engaging member 14 can have a variety of configurations, but in an exemplary embodiment it is adapted to engage one or more sutures that extend through the inner lumen 10c of the suture anchor 10. As shown in 7B, the suture-engaging member 14 is in the form of a post that extends transversely across the inner lumen 10c and between opposed inner sidewalls of the suture anchor 10. The angular orientation of the suture-engaging member 14 relative to a longitudinal axis A of the inner lumen 10c can vary, but in an exemplary embodiment the suture-engaging member 14 extends substantially perpendicular to the longitudinal axis A of the inner lumen 10c. The location of the suture-engaging member 14 can also vary, but in an exemplary embodiment the suture-engaging member 14 is positioned at or adjacent to the distal end 10b of the suture anchor 10. In the embodiment shown in FIG. 6B, the suture-engaging member 14 is located just proximal to the distal-most end 10b of the suture anchor 10 so as to form a suture-seating groove 22a in the distal-most end of the suture anchor 10. This recessed configuration of the suture-engaging member 14 can allow a suture(s) disposed around the suture-engaging member 14 to sit flush or sub-flush with the distal end 10b of the suture anchor 10 such that the suture(s) will not interfere with insertion of the suture anchor 10 into bone. A person skilled in the art will appreciate that the suture-engaging member 14 can be integrally formed with the suture anchor 10, i.e., the suture anchor 10 and suture-engaging member 14 can be molded as a single unit or formed from a single piece of material, or the suture-engaging member 14 can be fixedly or removably mated to the suture anchor 10.

As previously indicated, the suture anchors and inserters disclosed herein can be configured for use with one or more sutures. The particular quantity of sutures used with a suture anchor and inserter can depend on the size of the suture anchor and the inserter, and in particular on the diameter of the inner lumen of the suture anchor and on the diameter of the lumen in the inserter. While a single suture can be sufficient to anchor tissue to bone, it is preferred to use more than one, and more preferably two sutures. The one or more strands of suture can be loaded onto the suture anchor 10 in various ways. For example, in one embodiment the one or more strands of suture can be loaded onto the suture anchor 10 during manufacturing. In another embodiment, the one or more strands of suture can be threaded onto the suture anchor 10 by a user prior to deploying the suture anchor 10 into bone. As another example, the suture anchor 10 can be a knotless suture anchor 10 that does not require a surgeon to tie the one or more strands of suture into a knot after the anchor is deployed in a patient or the suture anchor 10, and the knotless anchor can automatically lock the strand(s) of suture in the anchor when the anchor is seated in the bone tunnel. Alternatively, the suture anchor 10 can be knotted during or after the anchor is seating in the bone tunnel in a patient's body.

The devices described above can be used to perform a surgical procedure for attaching soft tissue to bone. One skilled in the art will understand that the procedure is preferably a minimally invasive surgical procedure, but can alternatively be an open surgical procedure or a robotic-assisted surgical procedure. As one skilled in the art will appreciate, the procedure usually begins by preparing the patient for surgery and making one or more appropriately sized incisions at a desired location.

With or without the aid of imaging, tissue can be retracted if needed to provide a pathway to the bone. By way of non-limiting example, the bone can include the glenoid rim of a patient's shoulder. Once the pathway to the bone has been established, one or more strands of suture can be threaded through tissue to be reattached to the bone. A person skilled in the art will appreciate that the strand(s) of suture can be threaded through tissue either prior to or after insertion of the suture anchor 10 into bone, and this can depend on whether the strand(s) of suture are pre-loaded onto the suture anchor 10. A tunnel can be formed in the bone using various known techniques, such as using a bone drill that is advanced toward the bone, rotated and/or tapped to form the bone tunnel, and then removed from the body of the patient. In general, a size of the tunnel can be substantially equal to a size of the suture anchor 10, or the size of the tunnel can be smaller than the size of the anchor so that the suture anchor 10 can fit therein without large gaps between an outer surface of the suture anchor 10 and an inner surface of the tunnel. For example, a length of the tunnel along a longitudinal axis thereof can be substantially equal to or can be slightly less than a length of the suture anchor 10 from the proximal end to the distal tip. As another example, a diameter of the tunnel can be substantially equal to or can be slightly less than a diameter of the suture anchor 10. In both examples, such sizing can help prevent the suture anchor 10 from being dislodged from the tunnel during and/or after the surgical procedure.

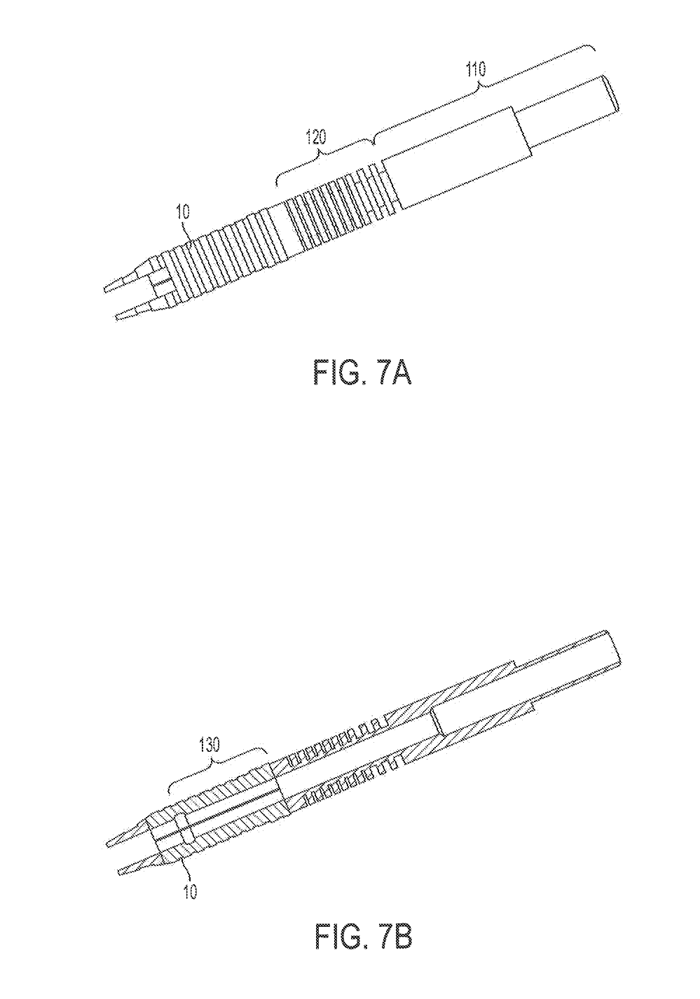

Before or after the tunnel is formed in the bone, the distal tip of the inserter can be releasably coupled to the suture anchor 10 in various ways. In general, this releasable coupling can be accomplished by inserting the distal tip of the inserter into the inner lumen formed in the suture anchor 10, as shown in FIGS. 7A and 7B. FIGS. 7A and 7B illustrate the inserter 100 and suture anchor 10 in a mated position, with the distal tip 130 of the inserter 100 positioned within the inner lumen 10c of the suture anchor 10. When the distal tip 130 is positioned inside of the suture anchor 10, the flexible distal portion 120 of the inserter 100 abuts and/or directly contacts the proximal end 10a of the suture anchor 10. When the strand(s) of suture are threaded through tissue and are threaded onto the suture anchor 10 prior to deploying the suture anchor 10 into the bone, the legs of the strand(s) of suture can extend through the inner lumen formed in the inserter and outside the patient's body.

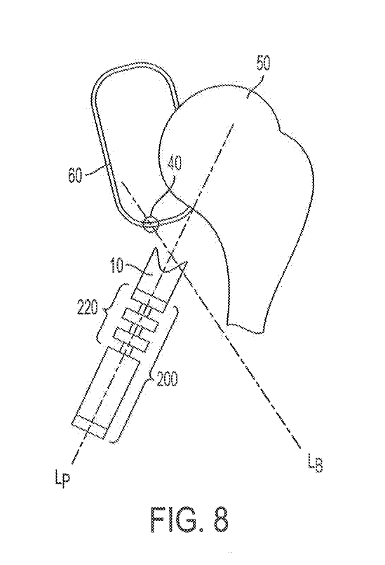

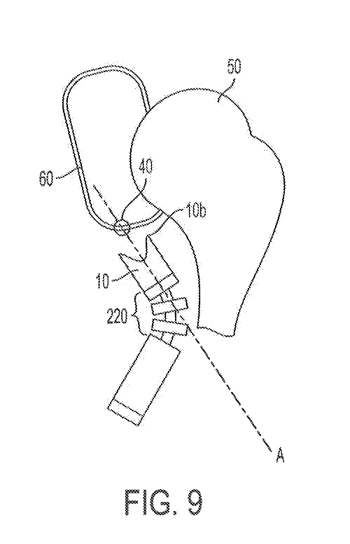

After the distal tip of the inserter is coupled to the suture anchor 10, the inserter and the suture anchor 10 can be advanced into the patient's body and toward a tunnel 40 formed in the bone. As shown in FIG. 8, one or more anatomical structures, such as a humeral head 50, can partially obstruct access to bone 60, such as a glenoid rim, such that a pathway L_{P} of the inserter 200 toward the bone 60 can be offset from and not-aligned with a longitudinal axis L_{B} of the bone tunnel, referred to herein as “off-axis insertion.” Because the inserter 200 cannot be inserted along a straight pathway toward the bone tunnel 40, the flexible portion 220 allows the inserter 200 to bend so that it curves around the anatomical structures, including the humeral head 50, allowing the suture anchor 10 to be inserted into the bone tunnel 40. During this off-axis insertion, the inserter 200 and/or the suture anchor 10 can be pressed against tissue or bone in the patient's body and this can cause the inserter 200 to first bend along the flexible distal portion 220. For example, when at least a portion of the distal tip 10b of the suture anchor 10 is pressed against and directly contacting the bone, and/or is positioned in the bone tunnel 40, a force can be applied to the anchor causing the flexible distal portion 220 to bend or flex to help align the longitudinal axis A of the suture anchor 10 with the longitudinal axis L_{B} of the tunnel 40, as shown in FIG. 9.

The flexing of the inserter during insertion of the anchor can occur in a variety of ways depending in part on the configuration of the distal portion of the inserter. For example, in embodiments in which the inserter has a plurality of projections and/or rings formed along the distal portion of inserter, bending of the distal portion will cause the rings on one side of the shaft to move toward one another. As the distal portion of the inserter continues to bend, the rings will eventually directly contact one another. The two proximal-most rings will contact one another first, followed in succession by the rings positioned distal to the two proximal-most rings. In embodiments in which the flexible portion of the inserter is heat treated and/or annealed, the heat treated and/or annealed sections of the inserter will function in a similar way, with bending occurring at proximal portion first and then continuing distally along the flexible distal portion of the inserter. Embodiments in which the inserter has a shaft diameter that varies along the flexible distal portion will also function in this way. More specifically, regions of the shaft at the proximal end of the flexible distal portion with a smaller diameter than regions of the shaft at the distal end of the distal portion will bend first, and the shaft will continue to bend in the proximal-to-distal direction until the rings contact adjacent distal rings in succession.

As previously mentioned, the inserter can be elastically bent or can be plastically deformed, and this can depend on the magnitude of the applied bending force and the type of material(s) used to form the inserter. For example, as previously described, the inserter can be formed from a shape memory material, such as Nitinol, that can automatically assume the straightened configuration after the bending force is no longer applied to the inserter. As another example, if the applied bending force exceeds the limits of the material forming the inserter, the inserter can be plastically deformed and will thus maintain the bent shape even after the bending force is no longer applied to the inserter. When such plastic deformation occurs, the method can also include applying an opposite bending force to the inserter, as this can move the inserter from the bent configuration to the straightened configuration.

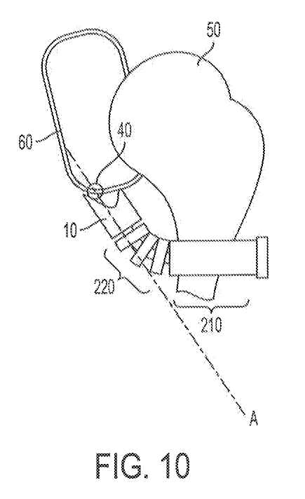

As the suture anchor 10 is being deployed into the bone tunnel, the inserter can be bent any number of times and in any number of directions to facilitate aligning the longitudinal axis of the suture anchor 10 with the longitudinal axis of the bone tunnel. For example, as the suture anchor 10 is advanced distally toward the bone tunnel 40, the inserter 200 can be bent in a first direction as shown in FIG. 9 and can then be bent in a second direction as shown in FIG. 10, the first direction being opposite to the second direction such that the proximal end of the inserter 200 moves along a single plane. As the inserter 200 is bent one or more times, the inserter can be advanced distally toward the bone 60 to drive the suture anchor 10 into the bone tunnel 40. In embodiments where the suture anchor 10 includes threads on an outer surface thereof, the inserter can be rotated to deploy the suture anchor 10 into the tunnel. Alternatively or additionally, the inserter can be used as a tap and a hammering force can be applied to the proximal portion 210 of the inserter to advance the suture anchor 10 into the bone tunnel. The suture anchor 10 can be advanced using any of the above described techniques until the suture anchor 10 is at least partially seated in the bone tunnel 40. In embodiments where the suture anchor 10 is threaded when the anchor 10 is positioned in a patient's body, the suture (not shown) can be coupled to the suture engaging member in the suture anchor 10 using known suture loading techniques, such as by threading the strand(s) of suture around the suture engaging member. The one or more strands of suture can also extend proximally through the inner lumen formed in the inserter and outside a patient's body. Preferably, when the suture anchor 10 is partially seated in the bone tunnel, legs of the suture extending outside of the patient's body can be pulled to move the tissue attached thereto toward the suture anchor 10 and thus, toward the bone, until the tissue is in the desired position. The inserter can be moved distally, and in embodiments where the suture anchor 10 is a knotless suture anchor 10, fully seating the anchor in the bone tunnel can lock the strand(s) of suture therein. When the suture anchor 10 is fully seated in the bone tunnel such that it is flush or sub-flush with a proximal surface of the bone, the inserter can be removed from the patient's body while the legs of the suture remain positioned outside the patient's body. Once soft tissue is positioned near or in direct contact with the bone, trailing ends of the suture can be secured together by known techniques and excess suture can be trimmed.

One skilled in the art will appreciate further features and advantages of the invention based on the above-described embodiments. Accordingly, the invention is not to be limited by what has been particularly shown and described, except as indicated by the appended claims. Further, although the systems, devices, and methods provided for herein are generally directed to surgical techniques, at least some of the systems, devices, and methods can be used in applications outside of the surgical field. All publications and references cited herein are expressly incorporated herein by reference in their entirety.

What is claimed is:

1. An inserter for driving a suture anchor into bone, comprising:
an elongate shaft having proximal and distal ends, and having a flexible portion extending along a length thereof adjacent to the distal end, the flexible portion including a plurality of grooves extending at least partially circumferentially around the shaft and spaced at a distance apart from one another along a longitudinal axis of the shaft; and
a distal tip extending distally from the distal end of the elongate shaft, the distal tip being configured to mate with a suture anchor.

2. The device of claim 1, wherein the flexible portion of the elongate shaft has a flexibility that varies in a proximal-to-distal direction.

3. The device of claim 1, wherein the flexible portion of the shaft has a diameter that varies from a proximal-to-distal direction.

4. The device of claim 1, wherein the flexible portion includes a plurality of rings extending at least partially circumferentially around the elongate shaft and defining the plurality of grooves therebetween.

5. The device of claim 4, wherein a central longitudinal axis of the elongate shaft is substantially aligned with a central longitudinal axis of the plurality of rings.

6. The device of claim 4, wherein the plurality of rings have equal sized diameters.

7. The device of claim 4, wherein the plurality of rings have diameters that vary in a proximal-to-distal direction.

8. The device of claim 4, wherein the plurality of rings are spaced at equal distances apart along the longitudinal axis of the elongate shaft.

9. The device of claim 1, wherein an outer diameter of the distal tip is less than an outer diameter of the elongate shaft such that the distal tip is configured to extend into an inner lumen in a suture anchor.

10. A suture anchor system, comprising:
a suture anchor having an elongate body with proximal and distal ends and an inner lumen extending at least partially therethrough; and
an inserter having an elongate shaft with proximal end and distal ends, the distal end including a tip portion configured to couple to the suture anchor to mate the suture anchor to the elongate shaft, and a flexible portion positioned proximal to the tip portion and configured to allow the elongate shaft to bend along the flexible portion during insertion of the suture anchor into bone.

11. The system of claim 10, wherein the inserter has an inner lumen extending between the proximal end and the tip portion for receiving a suture therethrough.

12. The system of claim 10, wherein the flexible portion of the inserter includes a plurality of grooves spaced at a distance apart along the elongate shaft.

13. The system of claim 10, wherein, when the inserter is mated to the suture anchor, the flexible portion of the elongate shaft abuts a proximal end of the suture anchor.

14. The system of claim 10, wherein the flexible portion of the inserter includes a plurality of rings extending at least partially circumferentially around the elongate shaft and spaced apart along a longitudinal axis of the elongate shaft.

15. The system of claim 10, wherein the suture anchor includes a suture-engaging member adapted to receive a suture therearound.

16. A method for attaching tissue to bone, comprising:
inserting a bone anchor coupled to a distal tip of an inserter shaft into a bone hole, the inserter shaft having a plurality of grooves that define a flexible region formed in a distal portion thereof at a location proximal to the distal tip and proximal to the suture anchor, the flexible region bending during insertion of the suture anchor into the bone hole.

17. The method of claim 16, wherein the bone anchor is inserted into the bone hole without advancing the bone anchor over a guide wire.

18. The method of claim 16, wherein the bone anchor is inserted into the bone hole without inserting the bone anchor inside of a guide member.

19. The method of claim 16, wherein the flexible region of the inserter shaft includes a plurality of rings having the grooves formed therebetween such that when the inserter shaft bends along the flexible region, at least some of the plurality of rings contact one another to limit a strain applied to the inserter shaft.

20. The method of claim 16, wherein the inserter plastically deforms as the bone anchor is inserted into the bone hole.

21. The method of claim 16, wherein inserting the bone anchor into the bone hole includes inserting the bone anchor into a glenoid rim.
